(19) United States
(12) Patent Application Publication
(10) Pub. No.: US 2025/0090515 A1
(43) Pub. Date: Mar. 20, 2025

(54) 1-Deoxynojirimycin To Mitigate Hepatic Ischemia- Reperfusion

(71) Applicant: KING FAISAL UNIVERSITY, Al-Ahsa (SA)

(72) Inventors: Maged Elsayed MOHAMED, Al-Ahsa (SA); Nancy Safwat YOUNIS, Al-Ahsa (SA)

(21) Appl. No.: 18/369,455

(22) Filed: Sep. 18, 2023

(51) Int. Cl. A61K 31/445; A61K 9/08; A61K 47/38; A61P 1/16

(52) U.S. Cl. CPC A61K 31/445; A61K 9/08; A61K 47/38; A61P 1/16

(57) ABSTRACT

A method of mitigating Hepatic Ischemia-Reperfusion (Hep I/R) in a subject includes administering to a subject in need thereof a therapeutically effective amount of a 1-Deoxynojirimycin (1-DNJ) composition. The 1-DNJ composition can effectively treat and mitigate Hep I/R at dose ranging from about 50 mg/kg/day to about 100 mg/kg/day. 1-DNJ reduced the level of one or more inflammatory mediators (e.g. TNF-α, IL-6, IL-10), reduced the level of one or more oxidative stress-related biomarkers (e.g. CAT, SOD, GST, GSH, MDA, NO), reduced the level of one or more hepatic function biomarkers (e.g. ALT, AST, ALP, LDH) and increased the level of IL-10.

### BACKGROUND

### 1. Field

The disclosure of the present patent application relates to Hepatic Ischemia-Reperfusion (Hep I/R) treatment, and particularly, to a method of mitigating Hep I/R by administering 1-Deoxynojirimycin (1-DNJ).

### 2. Description of the Related Art

Hepatic ischemia-reperfusion (Hep I/R) takes place clinically during different situations of liver pathologies such as following liver transplantation, liver resection, or hemorrhagic shock, which can lead to high morbidity and mortality. Additionally, during the H I/R condition, reactive oxygen species (ROS) are typically generated, causing hepatocytes to undergo inflammation and apoptosis cascades bringing about liver cell damage. As a response to hepatocytes' exposure to ROS, the liver undergoes complicated oxidative stress response status, which comprises the upregulation of defensive genes to limit hepatocytes' injury.

1-Deoxynojirimycin (1-DNJ) is a naturally occurring sugar analogue with unique bioactivities. It is found in mulberry leaves and silkworms, as well as in the metabolites of certain microorganisms, including Streptomyces and Bacillus. However, the content of 1-DNJ in these plants and insects is relatively low. 1-DNJ has been used in anti-diabetic, antioxidant, anti-inflammatory, and anti-obesity applications. 1-DNJ is a potent α-glucosidase inhibitor, and it possesses anti-hyperglycemic, anti-obesity, anti-viral, and anti-tumor properties. Some derivatives of 1-DNJ, like miglitol, miglustat and migalastat, have been applied clinically to treat diseases such as diabetes and lysosomal storage disorders. The combined treatment of ibuprofen with 1-DNJ has been shown to be effective to stop both microglial activation and, subsequently, dopaminergic neuronal degeneration in MPTP induced Parkinson's disease in mice resulting in neuroprotection.

Thus, a method for treating or preventing Hep I/R solving the aforementioned problems is desired.

### SUMMARY

The present subject matter relates to a method of mitigating Hepatic Ischmeia-Reperfusion (Hep I/R) in a subject, comprising administering to a subject in need thereof a therapeutically effective amount of 1-Deoxynojirimycin and a pharmaceutically acceptable carrier. In this regard, the methods can comprise administering a therapeutically effective amount of a 1-DNJ composition comprising 1-DNJ and a pharmaceutically acceptable carrier to the subject.

In an embodiment, the present subject matter relates to a method of also reducing the level of one or more inflammatory mediators in a subject comprising administering to a subject in need thereof a therapeutically effective amount of 1-DNJ and a pharmaceutically acceptable carrier, wherein the one or more inflammatory mediators are selected from the group consisting of TNF-α, IL-6, IL-1β, and a combination thereof.

In an embodiment, the present subject matter relates to a method of also increasing the level of IL-10 in a subject comprising administering to a subject in need thereof a therapeutically effective amount of 1-DNJ and a pharmaceutically acceptable carrier.

In an embodiment, the present subject matter relates to a method of also reducing the level of one or more oxidative stress-related biomarkers in a subject comprising administering to a subject in need thereof a therapeutically effective amount of 1-DNJ and a pharmaceutically acceptable carrier, wherein the one or more oxidative stress-related biomarkers are selected from the group consisting of CAT, SOD, GST, GSH, MDA, NO, and a combination thereof.

In an embodiment, the present subject matter relates to a method of also reducing the level of one or more hepatic function biomarkers in a subject comprising administering to a subject in need thereof a therapeutically effective amount of 1-DNJ and a pharmaceutically acceptable carrier, wherein the one or more hepatic function biomarkers are selected from the group consisting of ALT, AST, ALP, LDH, and a combination thereof.

These and other features of the present subject matter will become readily apparent upon further review of the following specification and drawings.

### BRIEF DESCRIPTION OF THE DRAWINGS

FIGS. 1A-1D depict graphs illustrating the effect of 1-DNJ (50 and 100 mg/kg) preconditioning for 2 weeks prior to Hep I/R on hepatic function biomarkers, including ALT (FIG. 1A), AST (FIG. 1B), ALP (FIG. 1C), and LDH (FIG. 1D). All values are stated as mean±SD (n=6). [symbol] designates statistically significant compared to the sham group and ¥ designates statistically significant compared to the H I/R group, Φ designates statistically significant compared to 1-DNJ 100+H I/R (p<0.05) using one-way ANOVA followed by Tukey's post hoc test.

FIGS. 2A-2F depicts graphs illustrating the effect of 1-DNJ (50 and 100 mg/kg) preconditioning for 2 weeks prior to Hep I/R on lipid peroxidation and antioxidant marker levels, including MDA (FIG. 2A), NO (FIG. 2B), SOD (FIG. 2C), CAT (FIG. 2D), GST (FIG. 2E), and GHS (FIG. 2F). All values are stated as mean±SD. [symbol] designates statistically significant compared to the sham group, ¥ designates statistically significant compared to the H I/R group, and Φ designates statistically significant compared to 1-DNJ 100+H I/R (p<0.05) using one-way ANOVA followed by Tukey's post hoc test.

FIGS. 3A-3E depict histopathological sections stained with hematoxylin and eosin showing the effect of 1-DNJ (50 and 100 mg/kg) preconditioning for 2 weeks prior to Hep I/R, including a control/sham treatment (FIG. 3A), a control/sham treatment with 1-DNJ (FIG. 3B), Hep I/R (FIG. 3C), Hep I/R with 50 mg/kg 1-DNJ (FIG. 3D), and Hep I/R with 100 mg/kg 1-DNJ (FIG. 3E).

FIGS. 4A-4E depict graphs illustrating the effect of 1-DNJ (50 and 100 mg/kg) preconditioning for 2 weeks prior to Hep I/R on inflammatory marker levels, including TNF-α (FIG. 4A), IL-10 (FIG. 4B), IL-6 (FIG. 4C), IL-10 (FIG. 4D), and NFκB (FIG. 4E). All values are stated as mean±SD. [symbol] designates statistically significant compared to the sham group, ¥ designates statistically significant compared to the H I/R group, and (designates statistically significant compared to 1-DNJ 100+H I/R (p<0.05) using one-way ANOVA followed by Tukey's post hoc test.

FIGS. 5A-5B depict graphs illustrating the effect of 1-DNJ (50 and 100 mg/kg) preconditioning for 2 weeks prior to Hep I/R on caspase 3 (FIG. 5A) and caspace 9 (FIG. 5B) levels. All values are stated as mean±SD. [symbol] designates statistically significant compared to the sham group, ¥ designates statistically significant compared to the H I/R group, and D designates statistically significant compared to 1-DNJ 100+H I/R (p<0.05) using one-way ANOVA followed by Tukey's post hoc test.

Similar reference characters denote corresponding features consistently throughout the attached drawings.

### DETAILED DESCRIPTION OF THE PREFERRED EMBODIMENTS

The following definitions are provided for the purpose of understanding the present subject matter and for construing the appended patent claims.

### Definitions

It should be understood that the drawings described above or below are for illustration purposes only. The drawings are not necessarily to scale, with emphasis generally being placed upon illustrating the principles of the present teachings. The drawings are not intended to limit the scope of the present teachings in any way.

Throughout the application, where compositions are described as having, including, or comprising specific components, or where processes are described as having, including, or comprising specific process steps, it is contemplated that compositions of the present teachings can also consist essentially of, or consist of, the recited components, and that the processes of the present teachings can also consist essentially of, or consist of, the recited process steps.

It is noted that, as used in this specification and the appended claims, the singular forms “a”, “an”, and “the” include plural references unless the context clearly dictates otherwise.

In the application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

In certain embodiments, a subject is a non-human mammal. Exemplary non-human mammals include laboratory, domestic, pet, sport, and stock animals, e.g., mice, cats, dogs, horses, and cows. As used herein, the term “patient” refers to any single subject for which treatment is desired. In certain embodiments, the patient herein is a human. A subject can be considered to be in need of treatment.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

Unless defined otherwise, all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language. However, it will be understood by one of skill in the art, that in some specific instances, an embodiment can alternatively be described using the language “consisting essentially of” or “consisting of”.

In an embodiment, the present subject matter relates to a method of treating and mitigating Hepatic Ischemia-Reperfusion (Hep I/R) in a subject, comprising administering to a subject in need thereof a therapeutically effective amount of a 1-DNJ composition comprising 1-Deoxynojirimycin and a pharmaceutically acceptable carrier. The 1-DNJ composition can be administered at an effective dose that ranges from about 50 mg/kg/day to about 100 mg/kg/day. The effective dose can be administered for a period of time in advance of an event that may increase the risk of a Hep I/R injury. In an embodiment, the period of time may be two weeks. In an embodiment, the 1-DNJ formulation is administered orally.

In an embodiment, the present subject matter relates to a method of also increasing the level of IL-10 in a subject comprising administering to a subject in need thereof a therapeutically effective amount of 1-Deoxynojirimycin and a pharmaceutically acceptable carrier.

In an embodiment, in addition to mediating HepI/R, the present subject matter relates to a method of also reducing the level of one or more inflammatory mediators in a subject comprising administering to a subject in need thereof a therapeutically effective amount of 1-Deoxynojirimycin and a pharmaceutically acceptable carrier, wherein the one or more inflammatory mediators are selected from the group consisting of TNF-α, IL-6, IL-1β, and a combination thereof.

In an embodiment, in addition to mediating HepI/R, the present subject matter relates to a method of also reducing the level of one or more oxidative stress-related biomarkers in a subject comprising administering to a subject in need thereof a therapeutically effective amount of 1-Deoxynojirimycin and a pharmaceutically acceptable carrier, wherein the one or more oxidative stress-related biomarkers are selected from the group consisting of CAT, SOD, GST, GSH, MDA, NO, and a combination thereof.

In an embodiment, in addition to mediating HepI/R, the present subject matter relates to a method of also reducing the level of one or more hepatic function biomarkers in a subject comprising administering to a subject in need thereof a therapeutically effective amount of 1-Deoxynojirimycin and a pharmaceutically acceptable carrier, wherein the one or more hepatic function biomarkers are selected from the group consisting of ALT, AST, ALP, LDH, and a combination thereof.

An embodiment of the present subject matter is directed to a pharmaceutical composition comprising 1-DNJ and a pharmaceutically acceptable carrier. In an embodiment, the pharmaceutically acceptable carrier may be carboxymethyl cellulose. In an embodiment, the pharmaceutical composition may comprise 1-DNJ suspended in a 1% carboxymethyl cellulose solution.

An embodiment of the present subject matter is directed to a method of making a pharmaceutical composition including mixing 1-DNJ with a pharmaceutically acceptable carrier. For example, the method of making a pharmaceutical composition can include mixing the 1-DNJ under sterile conditions with a pharmaceutically acceptable carrier with preservatives, buffers, and/or propellants to create the pharmaceutical composition.

To prepare the pharmaceutical composition, the 1-DNJ, as the active ingredient, can be intimately admixed with a pharmaceutically acceptable carrier according to conventional pharmaceutical compounding techniques. Carriers include inert pharmaceutical excipients, including, but not limited to, binders, suspending agents, lubricants, flavorings, sweeteners, preservatives, dyes, and coatings. In preparing compositions in an oral dosage form, any of the pharmaceutical carriers known in the art may be employed. For example, for liquid oral preparations, suitable carriers and additives include water, glycols, oils, alcohols, flavoring agents, preservatives, coloring agents, and the like. Further, for solid oral preparations, suitable carriers and additives include starches, sugars, diluents, granulating agents, lubricants, binders, disintegrating agents, and the like.

The present compositions can be in unit dosage forms such as tablets, pills, capsules, powders, granules, ointments, sterile parenteral solutions or suspensions, metered aerosol or liquid sprays, drops, ampules, auto-injector devices or suppositories, for oral parenteral, intranasal, sublingual or rectal administration, or for administration by injection, inhalation or insufflation. The nanocomposite formulation can be mixed under sterile conditions with a pharmaceutically acceptable carrier and, if required, any needed preservatives, buffers, or propellants. The composition can be presented in a form suitable for daily, weekly, or monthly administration. The pharmaceutical compositions herein will contain, per dosage unit, e.g., tablet, capsule, powder, injection, teaspoonful, suppository and the like, an amount of the active ingredient necessary to deliver an effective dose. A therapeutically effective amount of the 1-DNJ composition or an amount effective to treat or mitigate a disease, such as Hep I/R, may be determined initially from the Examples described herein and adjusted for specific targeted diseases using routine methods.

The 1-DNJ composition can be administered to a subject in need thereof. In an embodiment, the 1-DNJ composition can be administered to a subject in need thereof to treat or mitigate Hep I/R.

An embodiment of the present subject matter is directed to a method of treating and mitigate Hep I/R in a subject, comprising administering to a subject in need thereof a therapeutically effective amount of the pharmaceutical composition according to the present subject matter.

The 1-DNJ composition or pharmaceutical compositions thereof can be administered to a subject by any suitable route. For example, the compositions can be administered orally (including bucally and sublingually), nasally, rectally, intracisternally, intra vaginally, intraperitoneally, topically, transdermally (as by powders, ointments, or drops), and/or parenterally. As used herein, “parenteral” administration refers to modes of administration other than through the gastrointestinal tract, which include intravenous, intramuscular, intraperitoneal, intrasternal, intramammary, intraocular, retrobulbar, intrapulmonary, intrathecal, subcutaneous and intraarticular injection and infusion. Surgical implantation may also be contemplated, including, for example, embedding a composition of the disclosure in the body such as, for example, in a tissue, in the abdominal cavity, under the splenic capsule, brain, or in the cornea.

The present teachings are illustrated by the following examples.

### Example 1

### Materials and Methods

Wistar male rats (age: 6-8 weeks; weight: 200-230 g) were obtained from Experimental Animal Research Centre, King Saud University, Riyadh, K S A. The animals were acclimatized with standard laboratory food and water ad libitum in ventilated cages system (12 h light/dark cycles, 20.3-23.1° C.) then kept throughout the whole experiment under the same conditions.

All the experiments were performed in agreement with the relevant measures and guidelines of the Ethical Conduct for the Use of Animals in Research at King Faisal University. The Institutional Animal Care and Use Committee at King Faisal University permitted the experimental protocol under the number (KFU-REC-2022-FEB-EA000418).

Rats were anesthetized with isoflurane with oxygen (2%, 0.5 L/h) throughout the whole surgery, under heat control and aseptic conditions. Animals' abdomen was shaved and sterilized, and a surgical incision (about 3 cm) from the pubic to the sternum was executed. The portal triad, comprising the hepatic artery, portal vein, and common bile duct, was clamped using a micro-vascular clamp for 60 min to prompt ischemia. Hepatic reperfusion was accompanied by releasing the micro-vascular clamp and permitted reperfusion for six hours. The sham-operated animals were subjected to all the procedures that occurred during H I/R, deprived of blood flow blockage.

1-Deoxynojirimycin hydrochloride (1-DNJ) was purchased from Merck (Merck & Co., USA, product number D 9305). 1-DNJ was administered by gavage orally after being suspended in 1% carboxymethyl cellulose (CMC) solution. 1-DNJ dose (50 and 100 mg/kg) selection was based on previous studies. A total of 30 Wistar rats were haphazardly assigned into five groups (six rats/group). The sham group in which animals were given vehicle (1% CMC) orally for two weeks and subjected to sham steps. 1-DNJ+Sham group in which rats were administered 1-DNJ (100 mg/kg/day) for two weeks and then subjected to sham steps. Third group, the H I/R group, in which rats were administered 1% CMC for two weeks and then hepatic ischemia for 60 min followed by 6 hours reperfusion was implemented. Finally, 1-DNJ 50+H I/R and 1-DNJ 100+H I/R groups in which rats were pretreated with 1-DNJ (50, 100 mg/kg/day) respectively orally for two weeks, then subjected to hepatic I/R surgical procedure.

Once the reperfusion period was completed, animals' blood was obtained through the hearts, centrifuged at 4000×g for 15 min at 4° C., and stored at −20° C. to be utilized in the biochemical estimation of liver function tests, including ALT, AST, ALP, and LDH. Moreover, hepatic tissues were isolated, washed in cool PBS, and distributed in three portions. The first part was homogenized with PBS, centrifuged at 10,000×g for 15 min at 4° C., and consumed to evaluate oxidative stress and ELISA biomarkers. The second portion was stored at −20° C. for subsequent qRT-PCR and western blot analysis. The third part was fixed with 10% formalin buffered saline to estimate the histopathological variations as well as the immunohistochemical manifestation.

Data are demonstrated as mean±SD. One-way ANOVA followed by Tukey-Kramer as a post hoc test were executed for multiple comparisons between different experimental groups. p as a level of probability was set at 0.05, indicating the significance level. All statistical analyses were performed using Graph Pad software (version 8, CA, USA).

### Example 2

### Assessment of Hepatic Function Biomarkers

Rat Alanine Aminotransferase (ALT) ELISA kit (ab285264), Aspartate Aminotransferase (AST) ELISA kit (ab263883), and Alkaline Phosphatase (ALP) colorimetric assay kit (ab83369), Lactate Dehydrogenase (LDH) colorimetric assay kit (ab102526) were obtained from Abcam (Boston, MA, USA) and utilized following the manufacturer's instructions.

### 1-DNJ Alleviated Hepatic Function in H I/R-Induced Injury

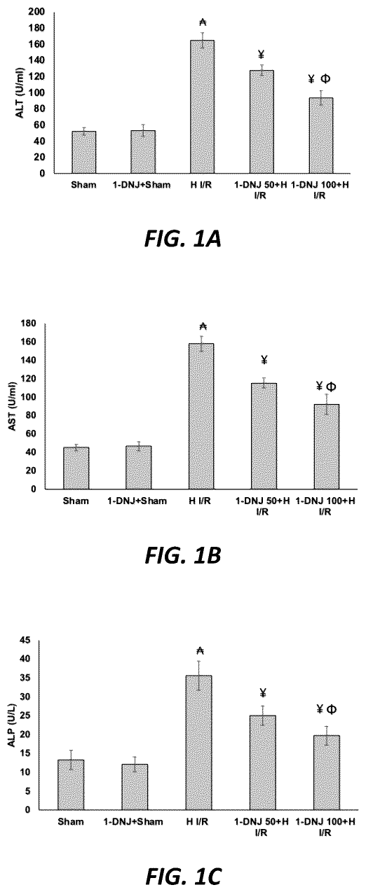

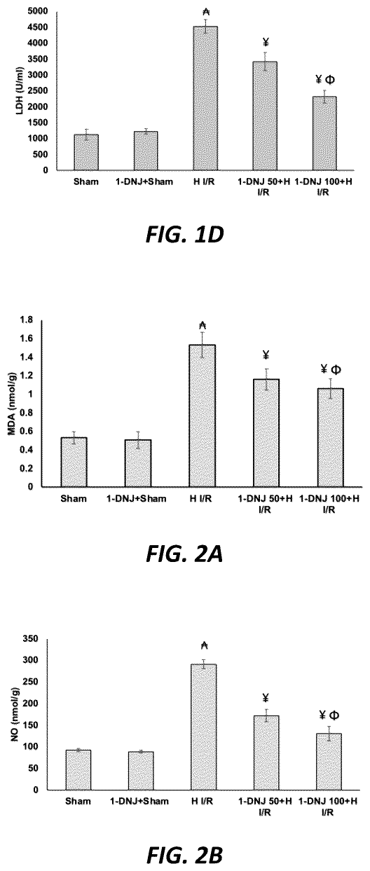

Hepatic function was evaluated by identifying the serum levels ALT, AST, ALP and LDH. As displayed in FIGS. 1A-1D, in comparison with the sham the H I/R animals exhibited elevated serum levels of ALT (FIG. 1A), AST (FIG. 1B), ALP (FIG. 1C), and LDH (FIG. 1D) (p<0.05). On the other hand, and compared with the H I/R group, 1-DNJ pretreatment alleviated ischemia-induced impairment in liver function, as confirmed by lowered serum levels of ALT, AST, ALP, and LDH (p<0.05). 1-DNJ (50 and 100 mg/kg) caused a percentage reduction in ALT (22.26% and 43.11%), AST (26.96% and 41.65%), ALP (29.75% and 44.53%) and LDH (24.39% and 48.63%) respectively, when compared to the H I/R animals.

### Example 3

### Assessment of Hepatic Oxidative Stress

Oxidative stress-related biomarkers, including antioxidant and lipid peroxidation enzymes such as CAT, SOD, GST, GSH, MDA, and NO contents, were monitored in the hepatic tissue homogenate. Malondialdehyde (MDA; ab238537), glutathione (GSH, ab65322), glutathione-s-transferase (GST, ab65326), nitric oxide (NO, ab272517) assay kits were purchased from Abeam Inc. (Cambridge, UK). Superoxide dismutase (SOD; MBS036924) and catalase (MBS726781) ELISA kits were obtained from My BioSource (San Diego, CA, USA). All the procedures were executed in accordance with the manufacturer's directions.

### 1-DNJ Alleviated Oxidative Stress in H I/R-Induced Injury

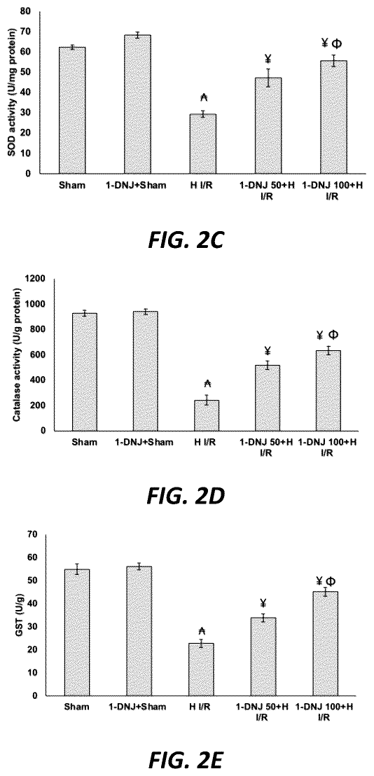

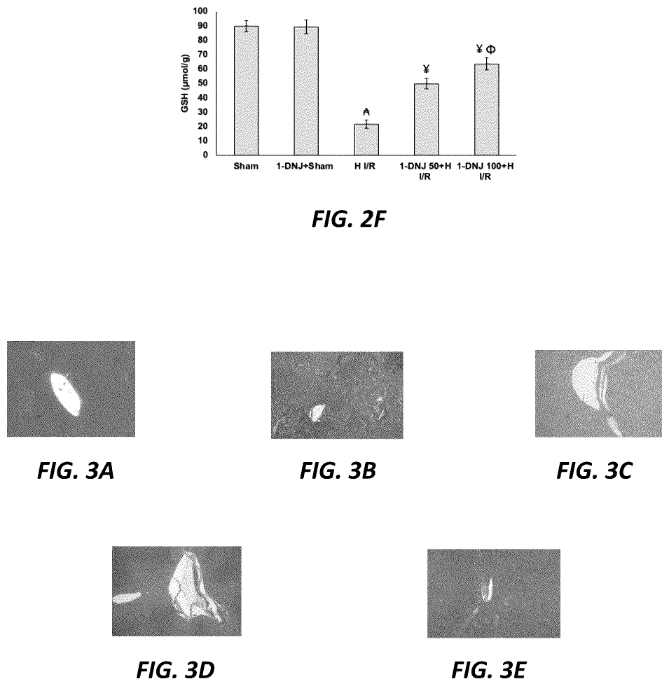

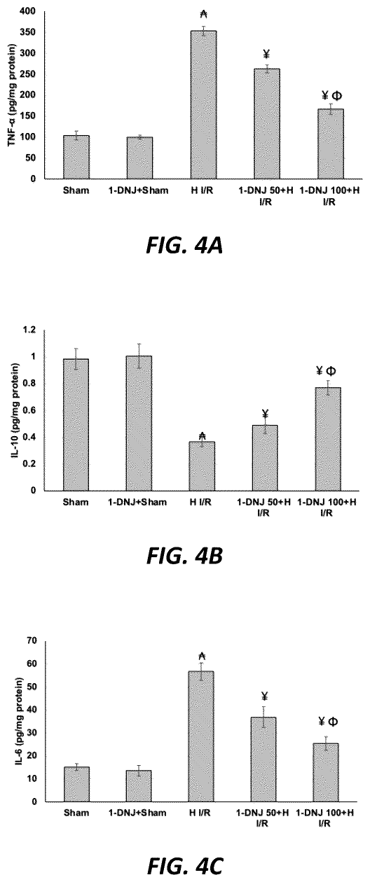

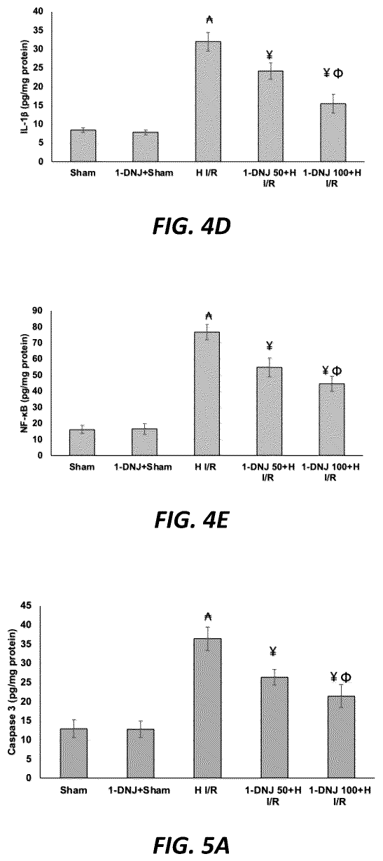

Several lipid peroxidation and antioxidant markers (MDA, NO, GSH, SOD, CAT, and GST) were evaluated in hepatic tissues. As illustrated in FIGS. 2A-2F, the ischemic animals exhibited elevated MDA (FIG. 2A), NO (FIG. 2B) contents and decreased activities of the anti-oxidant enzymes (SOD, FIG. 2C, CAT, FIG. 2D, and GST. FIG. 2E) and GSH (FIG. 2F) content. Alternatively, pretreatment with 1-DNJ diminished MDA, and NO contents and augmented the activities of the anti-oxidant enzymes (SOD, CAT, and GST) and GSH content. 1-DNJ (50 and 100 mg/kg) caused a percentage reduction in MDA (24.18% and 30.71%), NO (40.77% and 55.05%), and percentage elevation in GSH (130.82% and 194.64%) and SOD (60.80% and 89.51%), CAT (114.24% and 161.09%), GST (49.42% and 99.02%) respectively, when compared to the H I/R animals.

### Example 4

### Histopathological Examination Results

Part of the ischemic hepatic fraction was fixed with 10% formalin, embedded in paraffin, cut into 5 m thick paraffin slices, and stained with hematoxylin & eosin (H&E). Ischemic hepatic damage was estimated via two pathologists blinded to the investigational design under a light microscope (magnification, ×400) using Suzuki grading [4]. Suzuki grading standard includes five scores (0-4) related to hepatocyte cytoplasm vacuolization, necrosis, and tissue congestion.

In regards to the histopathological investigation, sham-operated groups showed regular histology architecture, while the H I/R animals exhibited plentiful histopathological variations, including diffuse necrosis with disorganized parenchyma and dilated sinusoids. 1-DNJ pretreated groups presented re-established hepatic parenchyma, less hepatocellular necrosis, fewer congested hepatic sinusoids, and portal vasculatures with slight vacuolation. (FIGS. 3A-3E) The histopathological changes were numerically expressed using Suzuki scores, where all the three features, congestion, vacuolation, and necrosis, were extensively amplified in the H I/R animals. While 1-DNJ preconditioned animals demonstrated a substantial reduction in hepatic congestion and hepatocellular vacuolation and necrosis when related to the H I/R animals.

### Example 5

### Assessment of Inflammation Mediators and Apoptotic Markers

Inflammation markers including TNF-α (ab46070), IL-10 (ab100768), IL-6 (ab100772) NFκB (ab133112) and IL-10 (ab214566), ELISA kits were obtained from Abcam Co., (Eugene, OR, USA). As for the apoptotic signaling markers, cleaved caspase-3 (KHO1091) was purchased from Thermo Fisher Scientific Inc. (Waltham, MA, USA), whereas caspase-9 (LS-F4141) was acquired from Biocompare (San Francisco, CA, USA). These markers were measured according to the manufacturer's instructions using a microplate reader SpectraMax i3X (Molecular devices San Jose, CA, USA).

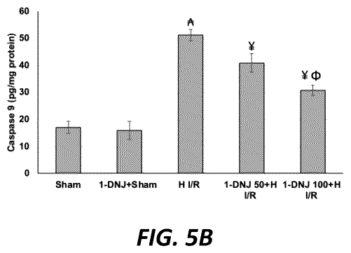

As for the apoptosis response, animals experimented with H I/R surgery displayed significantly exacerbated apoptotic markers, including caspase 3 and 9 levels (p<0.05). (See FIGS. 5A-5B) Pretreatment with 1-DNJ resulted in apoptosis moderation as established by the depressed caspase 3 (FIG. 5A) and caspase 9 (FIG. 5B) confirming the anti-apoptotic effect of 1-DNJ. 1-DNJ (50 and 100 mg/kg) caused a percentage reduction in caspase 3 (27.62% and 41.23%), caspase 9 (20.09% and 24.86%), respectively, when compared to the H I/R animals.

It is to be understood that the use of 1-DNJ to mitigate Hepatic Ischemia-Reperfusion is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A method of mitigating Hepatic Ischemia-Reperfusion in a mammal in need thereof, comprising administering to the mammal a 1-DNJ composition comprising 1-Deoxynojirimycin and a pharmaceutically acceptable carrier, the composition being administered in an amount of between about 50 mg/kg/day and about 100 mg/kg/day.

2. (canceled)

3. The method of claim 1, wherein the pharmaceutically acceptable carrier comprises carboxymethyl cellulose.

4. The method of claim 3, wherein the 1-DNJ is suspended in a 1% carboxymethyl cellulose solution.

5. The method of claim 1, wherein administration of the 1-DNJ composition to the mammal further reduces the level of one or more inflammatory mediators in the mammal, wherein the one or more inflammatory mediators are selected from the group consisting of TNF-α, IL-6, IL-1β, and a combination thereof.

6. The method of claim 1, wherein administration of the 1-DNJ composition to the mammal further increases the level of IL-10 in the mammal.

7. The method of claim 1, wherein administration of the 1-DNJ composition to the mammal further reduced the level of one or more oxidative stress-related biomarkers in the mammal, wherein the one or more oxidative stress-related biomarkers are selected from the group consisting of CAT, SOD, GST, GSH, MDA, NO, and a combination thereof.

8. The method of claim 1, wherein administration of the 1-DNJ composition to the mammal further reduces the level of one or more hepatic function biomarkers in the mammal, wherein the one or more hepatic function biomarkers are selected from the group consisting of ALT, AST, ALP, LDH, and a combination thereof.
